(19) United States
(12) Patent Application Publication
(10) Pub. No.: US 2025/0377346 A1
(43) Pub. Date: Dec. 11, 2025

(54) FLOATING OBJECT IMAGING DEVICE

(71) Applicant: SONY GROUP CORPORATION, Tokyo (JP)

(72) Inventors: Toru AMANO, Tokyo (JP); Satoru IWASAKI, Tokyo (JP); Keiichi HIRANO, Tokyo (JP); Toru NAGARA, Tokyo (JP); Takaki MAEDA, Ibaraki (JP); Akira ABE, Chiba (JP); Paul Nicholas JOHNS, Hampshire (GB)

(73) Assignee: SONY GROUP CORPORATION, Tokyo (JP)

(21) Appl. No.: 18/876,774

(22) PCT Filed: May 1, 2023
(86) PCT No.: PCT/JP2023/017059

§ 371 Date: Dec. 19, 2024

(30) Foreign Application Priority Data
Jul. 1, 2022 (JP) 2022-107272

(51) Int. Cl. G01N 33/00; G01N 21/94

(52) U.S. Cl. CPC G01N 33/0073; G01N 21/94; G01N 33/0022; G01N 33/0062

(57) ABSTRACT

Provided is a floating object imaging device capable of capturing and imaging a floating object by one device. First, a rotation drive unit rotates a rotating body so that a floating object capturing body is located in a capturing region (region facing an opening portion). As a result, the floating object floating in the air is captured by the floating object capturing body via the opening portion. Subsequently, the rotation drive unit rotates the rotating body so that the floating object capturing body that has captured the floating object is located in an imaging region (region facing an imaging unit). Subsequently, the imaging unit captures an image of the floating object capturing body that has captured the floating object and is located in the imaging region.

### TECHNICAL FIELD

The present disclosure relates to a floating object imaging device.

### BACKGROUND ART

Conventionally, for example, there has been proposed a system for analyzing air in which a filter captures floating objects floating in air using an air quality monitoring device, then the filter that has captured the floating objects is removed, and the filter is imaged using an image sensor (See, for example, Patent Document 1.). In the system described in Patent Document 1, a plurality of regions on the filter is imaged, a plurality of pieces of image data obtained by the imaging is analyzed, and the number of floating objects is counted.

### CITATION LIST

### Patent Document

Patent Document 1: Japanese Patent Application Laid-Open No. 2021-522487

### SUMMARY OF THE INVENTION

### Problems to be Solved by the Invention

However, in the system described in Patent Document 1, a human performs work of removing the filter from the air quality monitoring device and setting the removed filter in a microscope.

An object of the present disclosure is to provide a floating object imaging device capable of capturing and imaging a floating object by one device.

### Solutions to Problems

A floating object imaging device of the present disclosure includes: (a) a housing including a recess; (b) a rotating body that is disposed within the recess and rotates about an axis extending in a depth direction of the recess; (c) a plurality of floating object capturing bodies including a material capable of capturing a floating object floating in air and disposed on an outer peripheral surface of the rotating body at intervals in a circumferential direction; (d) a rotation drive unit that is disposed in the housing and rotates the rotating body about the axis; (e) an opening portion formed in the housing at a position facing a first region on a rotation path of the floating object capturing bodies so that the floating object capturing bodies located in the first region are exposed to an outside; (f) an imaging unit disposed in the housing at a position facing a second region on the rotation path of the floating object capturing bodies so that the floating object capturing bodies located in the second region are imaged; and (g) a control unit that causes the rotation drive unit to rotate the rotating body so that the floating object capturing bodies are located in the second region after being located in the first region, and causes the imaging unit to image the floating object capturing bodies located in the second region.

### BRIEF DESCRIPTION OF DRAWINGS

FIG. 1 is a diagram illustrating an overall configuration of a floating object imaging device according to an embodiment, and is a diagram illustrating a case where the floating object imaging device is viewed from a front surface side.

FIG. 2 is a diagram illustrating the floating object imaging device in a case where a rotating body of FIG. 1 is removed.

FIG. 3 is a diagram illustrating the floating object imaging device of FIG. 1 and a lid that covers a recess.

FIG. 4 is a diagram illustrating an overall configuration of the floating object imaging device according to the embodiment, and is a diagram illustrating a case where the floating object imaging device is viewed from a back surface side.

FIG. 5 is a diagram illustrating the floating object imaging device of FIG. 4 and a back lid that covers a back surface.

FIG. 6 is a diagram illustrating a shutter mechanism, and is a diagram illustrating a case where a shutter is located at a lowermost portion of a movable range.

FIG. 7 is a diagram illustrating the shutter mechanism, and is a diagram illustrating a case where the shutter is located at an uppermost portion of the movable range.

FIG. 8 is an enlarged view of a part of the floating object imaging device of FIG. 2, and is a view illustrating a drain hole.

FIG. 9 is a diagram illustrating a floating object capturing body and a frame body.

FIG. 10 is a diagram illustrating identification numbers attached to floating object capturing bodies.

FIG. 11 is a diagram illustrating a combination pattern of positioning markers.

FIG. 12 is a diagram illustrating an illumination mechanism in a case where the rotating body is broken.

FIG. 13 is a diagram illustrating an internal configuration of a control unit.

FIG. 14 is a flowchart of a floating object imaging process.

FIG. 15 is a diagram illustrating a floating object imaging device according to a modification.

FIG. 16 is a diagram illustrating an internal configuration of a control unit according to the modification.

### MODE FOR CARRYING OUT THE INVENTION

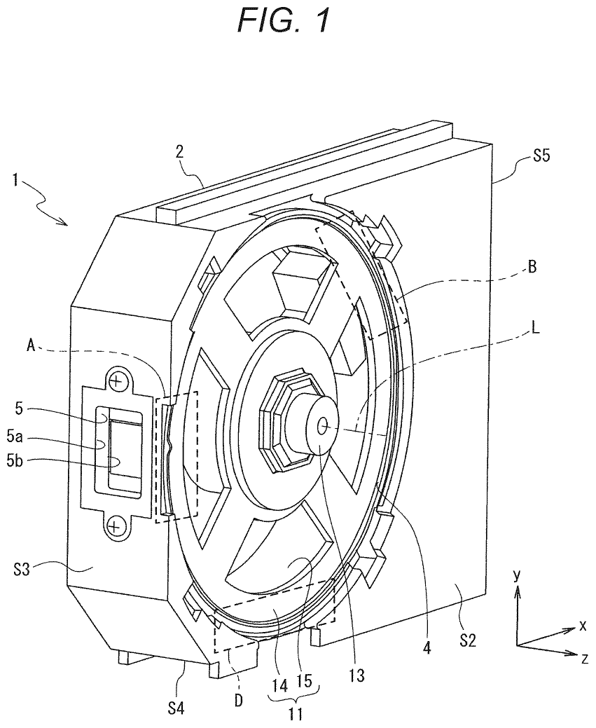

Hereinafter, an example of a floating object imaging device according to an embodiment of the present disclosure will be described with reference to FIGS. 1 to 16. Embodiments of the present disclosure will be described in the following order. Note that, the present disclosure is not limited to the following examples. Furthermore, the effects described in the present specification are illustrative and not restrictive, and there may be additional effects.
1. Embodiment: Floating object imaging device
1-1 Configuration of floating object imaging device
1-2 Floating object imaging process
1-3 Modifications

### 1. Embodiment: Floating Object Imaging Device

### 1-1 Configuration of Floating Object Imaging Device

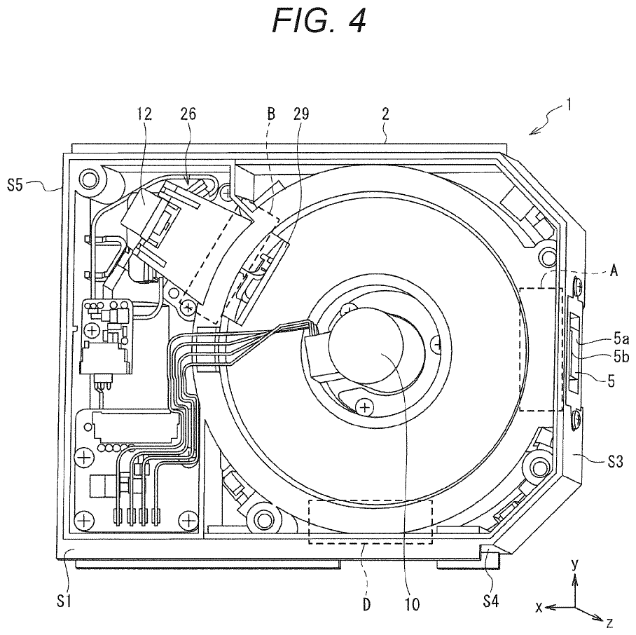

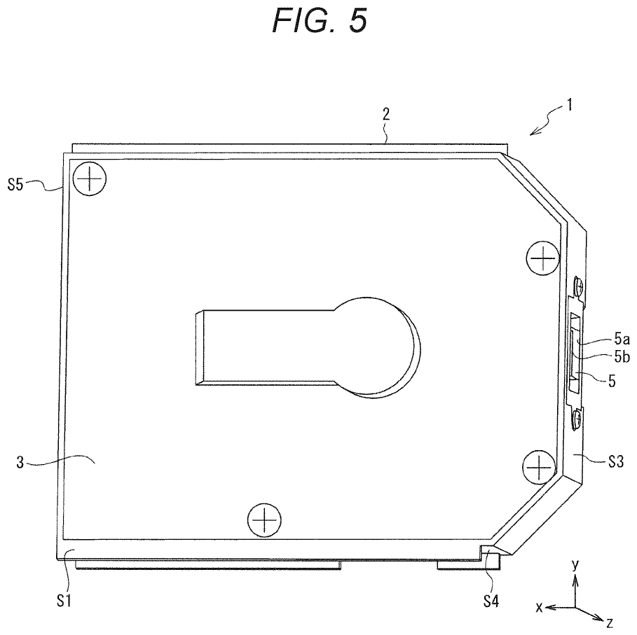

A floating object imaging device 1 according to an embodiment of the present disclosure will be described. FIGS. 1 to 5 are diagrams illustrating an overall configuration of a floating object imaging device 1 according to a first embodiment. FIGS. 1 to 3 illustrate a case where the floating object imaging device 1 is viewed from the front surface side, and FIGS. 4 and 5 illustrate a case where the floating object imaging device 1 is viewed from the back surface side. The floating object imaging device 1 is a device that captures a floating object floating in the air with a floating object capturing body and captures an image of the floating object capturing body that has captured the floating object to obtain image data. Examples of the floating object include pollen, spores, and insects floating in the air of a farm, bacteria and viruses floating in the air outdoors or indoors, and asbestos floating in the air at a dismantling site.

As illustrated in FIGS. 1 and 4, the floating object imaging device 1 includes a flat plate-shaped housing 2 to which each component of the floating object imaging device 1 is attached. Hereinafter, a direction along one side (one side of the flat plate) of the flat plate-shaped floating object imaging device 1 is referred to as an “x direction”, a direction along the other side (the other side of the flat plate) is referred to as a “y direction”, and a thickness direction (thickness direction of the flat plate) of the flat plate-shaped floating object imaging device 1 is referred to as a “z direction”. In a case where air flows in one direction in the horizontal direction, the housing 2 is disposed such that the x direction is parallel to the air flow direction, the y direction is parallel to the vertical direction, and the z direction is a horizontal direction orthogonal to the x direction. Note that, in the present embodiment, the “horizontal direction” includes not only a direction orthogonal to the direction in which gravity acts but also a direction (for example, a direction of ±15° in a direction orthogonal to a direction in which gravity acts.) considering an error range allowed at the time of installation of the floating object imaging device 1. FIGS. 1 and 4 exemplify a case where the shape of the housing 2 is a hexagonal (hexagon obtained by chipping two corners located at an end of one short side of a rectangle from the rectangle) flat plate shape.

As illustrated in FIG. 5, a back lid 3 is screwed to one principal surface (hereinafter, also referred to as a “back surface S1”) of the housing 2 so as to cover the components disposed on the back surface S1 side and the back surface S1 itself.

Furthermore, as illustrated in FIGS. 1 to 3, a bottomed circular recess (hereinafter, also referred to as a “recess 4”) having a circular opening is formed on the other principal surface (Hereinafter, also referred to as a “surface S2”.) of the housing 2 so as to be able to accommodate a cylindrical rotating body 11. A depth direction of the recess 4 is parallel to the z direction (horizontal direction). The recess 4 includes a columnar space having a constant gap with an outer peripheral surface of the rotating body 11 in a case where the rotating body 11 is accommodated in the recess 4. As a result, the rotating body 11 can be rotated about an axis L extending in the z direction within the recess 4.

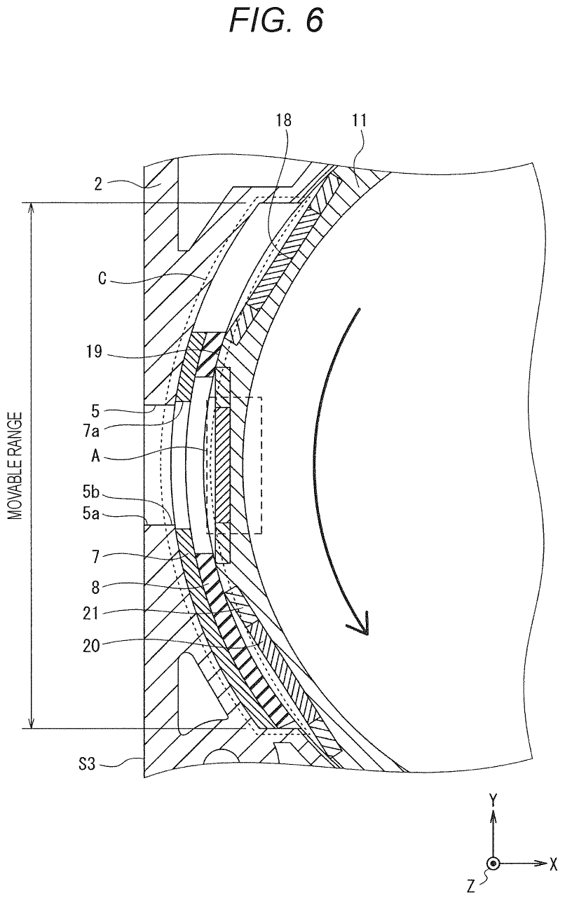

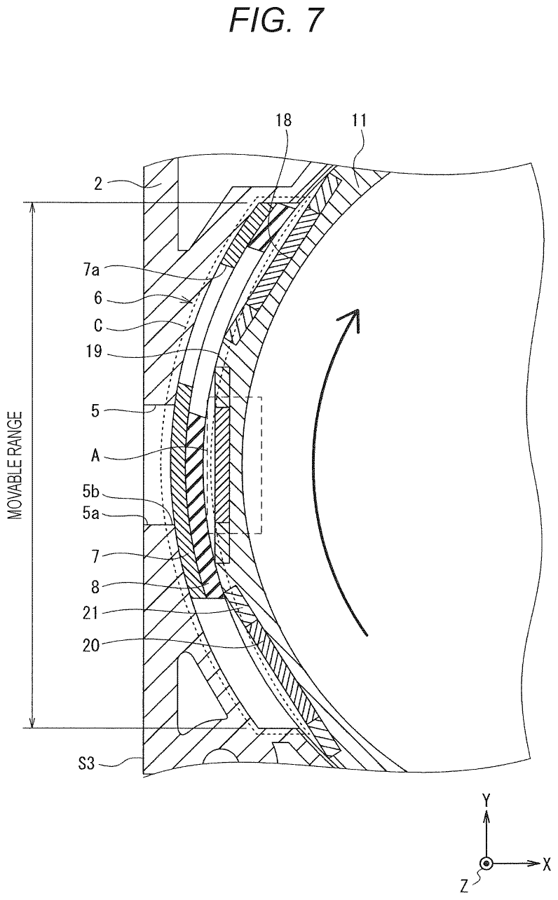

Furthermore, as illustrated in FIGS. 6 and 7, a through hole (hereinafter, also referred to as an “opening portion 5”) is formed on a side surface (hereinafter, also referred to as a “side surface S3”) opposite to the x direction of the housing 2 so that a floating object capturing body 20 located in a capturing region A (in a broad sense, “first region”) on a rotation path of the floating object capturing body 20 is exposed to the outside. As a result, the outside air is allowed to pass through, and the passing air can be brought into contact with the floating object capturing body 20 located in the capturing region A. FIGS. 6 and 7 illustrate a case where the opening portion 5 is formed at a position facing the floating object capturing body 20 located in the capturing region A in the housing 2. The opening portion 5 includes one opening 5a on the side surface S3 and the other opening 5b on the inner peripheral surface of the recess 4, and is a through hole having a rectangular cross section extending in the x direction (horizontal direction). An opening direction of the opening portion 5 extends in a direction orthogonal to the axis L which is the rotation center of the rotating body 11. That is, the axis L extends in the other horizontal direction orthogonal to the one horizontal direction in which the axis L extends. FIGS. 1 to 3 illustrate a case where the axis L and the center of the opening portion 5 are located at the same height, the axis L extends in the z direction, and a processing direction of the opening portion 5 extends in the x direction. Furthermore, FIGS. 1 to 3 illustrate a case where the opening portion 5 is formed by fitting a component separate from the housing 2, and FIGS. 6 and 7 illustrate a case where the opening portion 5 is directly formed in the housing 2.

Furthermore, as illustrated in FIGS. 6 and 7, a shutter mechanism 6 that opens and closes the opening 5b in conjunction with the rotational movement of the rotating body 11 is formed in a peripheral region C of the opening 5b of the opening portion 5 on the inner peripheral surface of the recess 4. The shutter mechanism 6 includes, for example, a shutter 7 that slides and moves in the circumferential direction on the inner peripheral surface of the recess 4 of the housing 2, and a friction component 8 that is attached to the surface of the shutter 7 on the rotating body 11 side and comes into contact with a portion (Hereinafter, also referred to as a “frame-shaped region 19”) surrounding the recessed region 18 in the outer peripheral surface of the rotating body 11. The frame-shaped region 19 includes a region between the adjacent recessed regions 18 and a region extending along the circumferential direction of the rotating body 11. The radius of the inner peripheral surface of the peripheral region C (region where the shutter 7 slides and moves) on the inner peripheral surface of the recess 4 is larger than the radius of the inner peripheral surface of the other regions. That is, the peripheral region C is recessed in the radial direction more than the other regions. Furthermore, the shutter 7 is a plate member curved in an arc shape along the recess of the peripheral region C. The length of the shutter 7 in the y direction (In FIGS. 6 and 7, the vertical direction) is shorter than the length of the peripheral region C in the y direction.

As a result, the shutter 7 is movable in the y direction (vertical direction) in the recess of the inner peripheral surface of the recess 4 in the peripheral region C. A movable range of the shutter 7 in the y direction is restricted by a difference between a length of the shutter 7 in the y direction and a length of the peripheral region C in the y direction. FIG. 6 is a diagram illustrating a case where the shutter 7 is moved to the lowermost portion of the movable range. Furthermore, FIG. 7 is a diagram illustrating a case where the shutter 7 is moved to the uppermost portion of the movable range. As a material of the shutter 7, for example, an ABS resin (acrylonitrile, butadiene, styrene copolymer synthetic resin), a PC resin (polycarbonate resin), or a PC-ABS resin can be adopted. Furthermore, as a material of the friction component 8, for example, silicone rubber, fluororubber, thermoplastic elastomer, or urethane cushion can be adopted. In particular, fluororubber is preferable from the viewpoint of the coefficient of friction.

Furthermore, as illustrated in FIG. 6, an opening portion 7a is formed in the shutter 7 at a position overlapping the opening 5b of the opening portion 5 in a case where the shutter 7 is moved to the lowermost portion of the movable range. An opening area of the opening portion 7a is larger than an opening area of the opening 5b. As a result, as illustrated in FIG. 6, when the shutter 7 is moved to the lowermost portion of the movable range, the opening portion 7a of the shutter 7 overlaps the opening 5b of the opening portion 5, and the opening 5b of the opening portion 5 is opened. On the other hand, as illustrated in FIG. 7, when the shutter 7 is moved to the uppermost portion of the movable range, a portion other than the opening portion 7a of the shutter 7 overlaps the opening 5b of the opening portion 5, and the opening 5b of the opening portion 5 is closed.

The friction component 8 covers the entire surface of the shutter 7 on the side of the rotating body 11. The thickness of the friction component 8 is adjusted such that only the frame-shaped region 19 (region other than the recessed region 18) of the outer peripheral surface of the rotating body 11 is in contact with the friction component 8. As a result, as illustrated in FIG. 6, in a case where the rotating body 11 is rotated counterclockwise as viewed from a surface S2 side, a downward force is applied to the shutter 7 by the frictional force between the frame-shaped region 19 of the rotating body 11 and the friction component 8, and the shutter 7 can be moved downward. Then, the opening 5b of the opening portion 5 can be opened by moving the shutter 7 to the lowermost portion. Therefore, when capturing and imaging the floating object, the rotation direction of the rotating body 11 is limited to the counterclockwise direction, so that the floating object floating in the air can enter the recessed region 18 of the rotating body 11 (region where the floating object capturing body 20 is disposed) from the opening portion 5.

On the other hand, as illustrated in FIG. 7, in a case where the rotating body 11 is rotated clockwise as viewed from the surface S2 side, a force in the upward direction is applied to the shutter 7 by the frictional force between the frame-shaped region 19 of the rotating body 11 and the friction component 8, and the shutter 7 can be moved upward. Then, the opening 5b of the opening portion 5 can be closed by moving the shutter 7 to the uppermost portion. Therefore, when neither capturing nor imaging of the floating object is performed, by limiting the rotation direction of the rotating body 11 to the clockwise direction, it is possible to prevent foreign matter, rain, or the like from entering the recessed region 18 of the rotating body 11 from the opening portion 5.

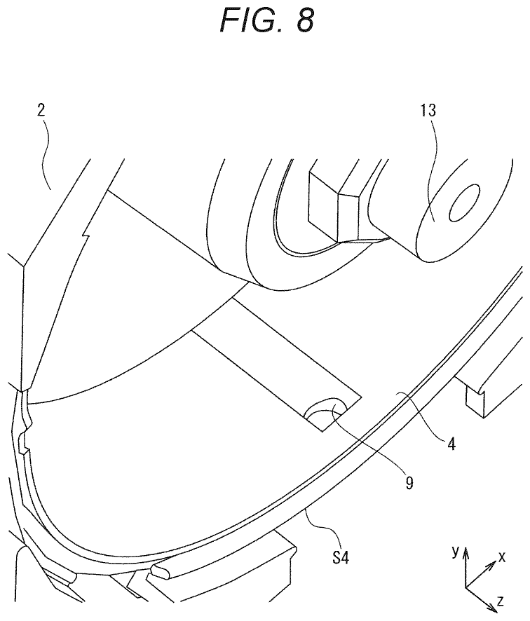

Furthermore, as illustrated in FIG. 8, a through hole (Hereinafter, also referred to as a “drain hole 9”.) penetrating from the bottom side of the inner peripheral surface to the outside of the housing 2 (lower surface S4 side) is formed in a lower region of the inner peripheral surface of the recess 4. As a result, in a case where the rotating body 11 is rotated counterclockwise as viewed from the surface S2 side at the time of capturing and imaging the floating object, foreign matter, rain, or the like that has entered the recessed region 18 of the rotating body 11 from the opening portion 5 can be dropped by gravity, and the dropped foreign matter, rain, or the like can be discharged to the outside of the housing 2 from the drain hole 9. FIG. 8 illustrates a case where a groove inclined from a side of the bottom portion to a side of the opening portion of the recess 4 is provided in a lower region in the recess 4, and the drain hole 9 is formed at the lower end of the groove.

Furthermore, as illustrated in FIGS. 1 to 4, the floating object imaging device 1 includes a rotation drive unit 10, a rotating body 11, and an imaging unit 12.

As illustrated in FIG. 4, the rotation drive unit 10 is disposed at a position overlapping a central portion of the recess 4 on a back surface S1 side of the housing 2 in a case of being viewed from the back surface S1 side of the housing 2. The rotation drive unit 10 includes a shaft portion 13 extending in the z direction (the depth direction of the recess 4; the horizontal direction) so that its tip portion protrudes into the recess 4, and is capable of rotating the shaft portion 13 about the axis L in accordance with a signal from the control unit 30. As a result, the rotation drive unit 10 can rotate the rotating body 11 about the axis L. As the rotation drive unit 10, for example, a stepping motor can be employed.

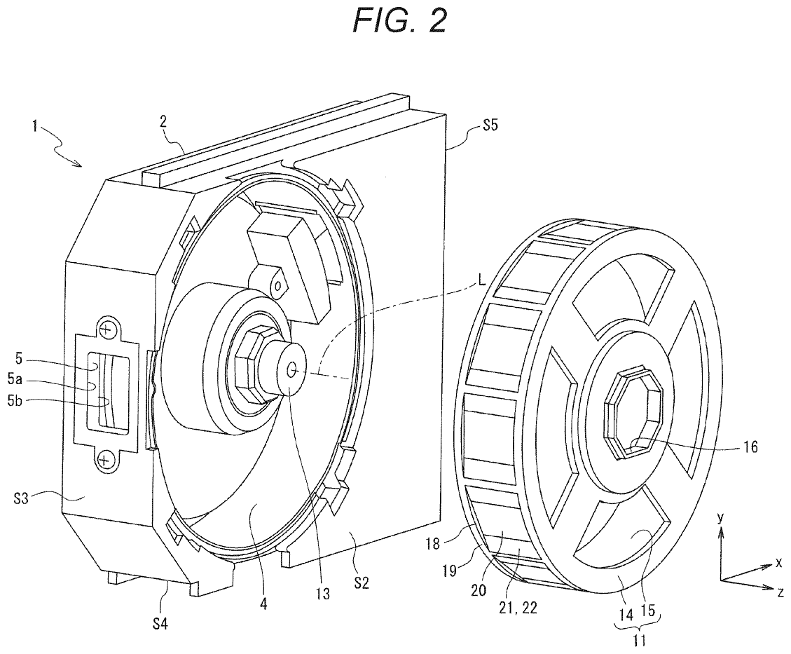

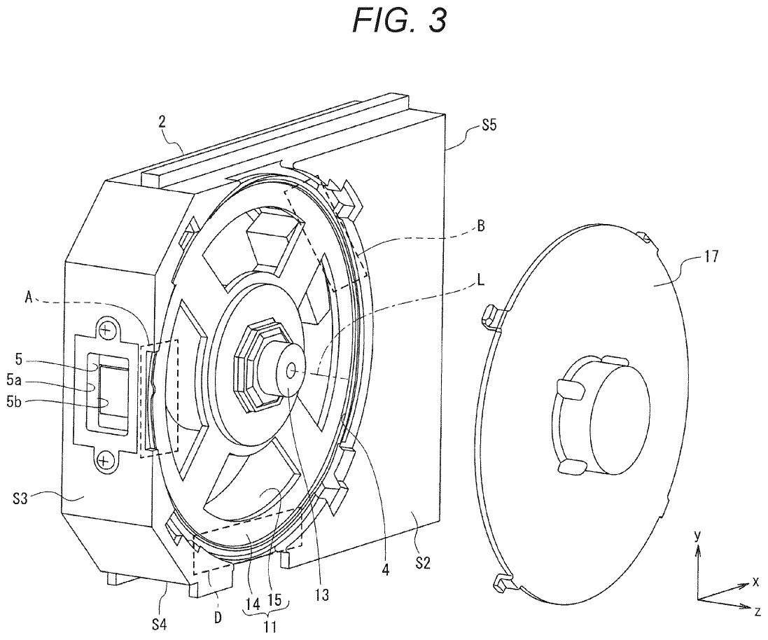

As illustrated in FIG. 2, the rotating body 11 has a disk-shaped bottom portion 14 and a cylindrical outer peripheral portion 15, and is formed in a bottomed cylindrical shape. The rotating body 11 is accommodated in the recess 4 such that the bottom portion 14 is positioned on the side of the opening portion of the recess 4 and the outer peripheral portion 15 faces the inner wall surface of the recess 4. A through hole 16 is formed in the center portion of the bottom portion 14 so that the tip portion of the shaft portion 13 penetrates in a case where the rotating body 11 is accommodated in the recess 4. As illustrated in FIG. 3, the rotating body 11 accommodated in the recess 4 is sandwiched between a lid 17 and the shaft portion 13 and fixed to the shaft portion 13 by attaching the lid 17 to the opening portion of the recess 4 so as to cover the space in the recess 4. As a result, the rotating body 11 is configured to be detachable from the shaft portion 13 of the rotation drive unit 10. Therefore, by replacing the rotating body 11, the floating object capturing body 20 can be easily replaced, and the trouble required for replacing the floating object capturing body 20 can be reduced. For example, in a case where the type of the floating object to be captured and imaged is changed, it is possible to replace the rotating body 11 with the floating object capturing body 20 according to the type of the floating object. Furthermore, by replacing the rotating body 11, it is also possible to continuously capture and image the floating object.

Furthermore, a plurality of recessed regions 18 formed at intervals in the circumferential direction and a frame-shaped region 19 surrounding the recessed regions 18 are formed on the outer peripheral surface of the outer peripheral portion 15. The frame-shaped region 19 can reduce a gap between the inner peripheral surface of the recess 4 of the housing 2 and the outer peripheral surface of the outer peripheral portion 15, and in a case where foreign matter, rain, or the like enters a certain recessed region 18, movement of the foreign matter, rain, or the like from the recessed region 18 to the adjacent recessed region 18 can be suppressed. FIG. 2 illustrates a case where 12 recessed regions 18 are formed. The recessed region 18 is formed along a central portion in a width direction (a direction orthogonal to the circumferential direction) of the outer peripheral portion 15, and has a flat plate shape with a bottom surface having a normal line extending in a direction orthogonal to the axis L. The floating object capturing body 20 is disposed in each of the recessed regions 18 (bottom surface). As a result, a plurality of floating object capturing bodies 20 is disposed on the outer peripheral surface of the outer peripheral portion 15 at intervals in the circumferential direction. Furthermore, the floating object capturing body 20 has a flat plate shape having a normal line extending in a direction orthogonal to the axis L. The floating object capturing body 20 includes a material capable of capturing a floating object floating in the air. As the floating object capturing body 20, for example, a capturing film or a capturing filter can be adopted. As the capture film, for example, a synthetic resin film that adsorbs or reacts a floating object to capture the floating object can be employed. Furthermore, as the capture filter, for example, a filter having finer meshes than the floating object (gauze, synthetic resin filter, glass fiber filter, etc.) can be adopted.

Furthermore, as the plurality of floating object capturing bodies 20 disposed on the outer peripheral surface of the outer peripheral portion 15, for example, the same type of floating object capturing bodies can be adopted. Furthermore, it is also possible to adopt a configuration including a plurality of types of floating object capturing bodies having different materials. In a case where the same kind of floating object capturing body is used for all the floating object capturing bodies, it is possible to capture the floating object capturing body 20 by dividing time by rotating the rotating body 11 such that the floating object capturing body positioned in the capturing region A is switched every predetermined time. Moreover, it is also possible to use the floating object capturing body 20 having an expiration date such as 30 minutes from the start of use. Furthermore, in a case where the configuration including a plurality of types of floating object capturing bodies is used, the floating object capturing body 20 according to the type of the floating object to be captured and imaged can be used without replacing the entire rotating body 11.

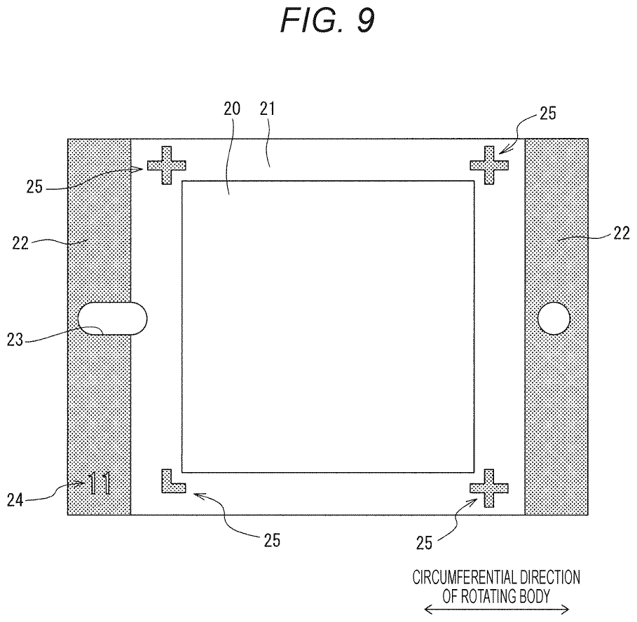

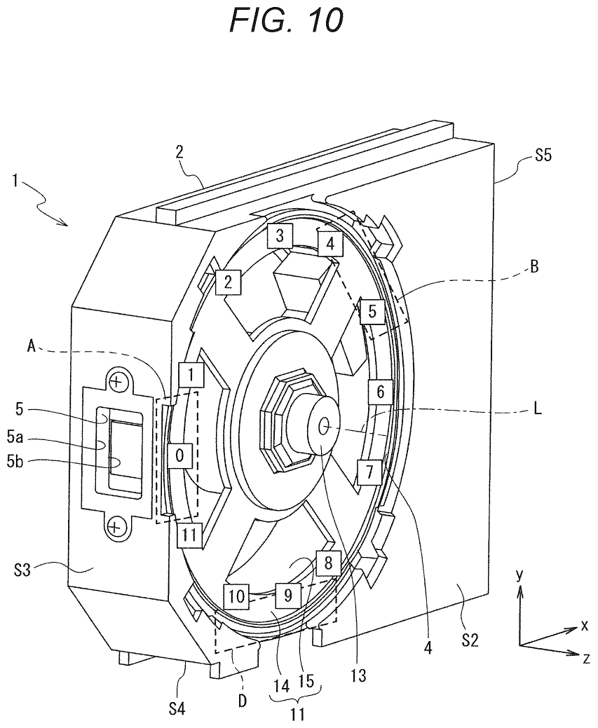

Furthermore, as illustrated in FIG. 9, a frame body 21 surrounding the floating object capturing body 20 is formed around the floating object capturing body 20. FIG. 9 illustrates a case where the floating object capturing body 20 has a rectangular shape and the frame body 21 has a rectangular frame shape. At end portions 22 of the frame body 21 (end portions located on the circumferential direction side of the rotating body 11; left and right ends in FIG. 9), a positioning hole 23 and an identification number 24 are formed. The identification number 24 is a number for identifying the floating object capturing body 20, and as illustrated in FIG. 10, “0”, “1”, “2”, . . . , and “11” are attached to each of the floating object capturing bodies 20. In FIG. 10, “0” to “11” are sequentially attached to each of the floating object capturing bodies 20 so that the identification number 24 increases clockwise by “1” in a case where the rotating body 11 is accommodated in the recess 4 and viewed from the surface S2 side.

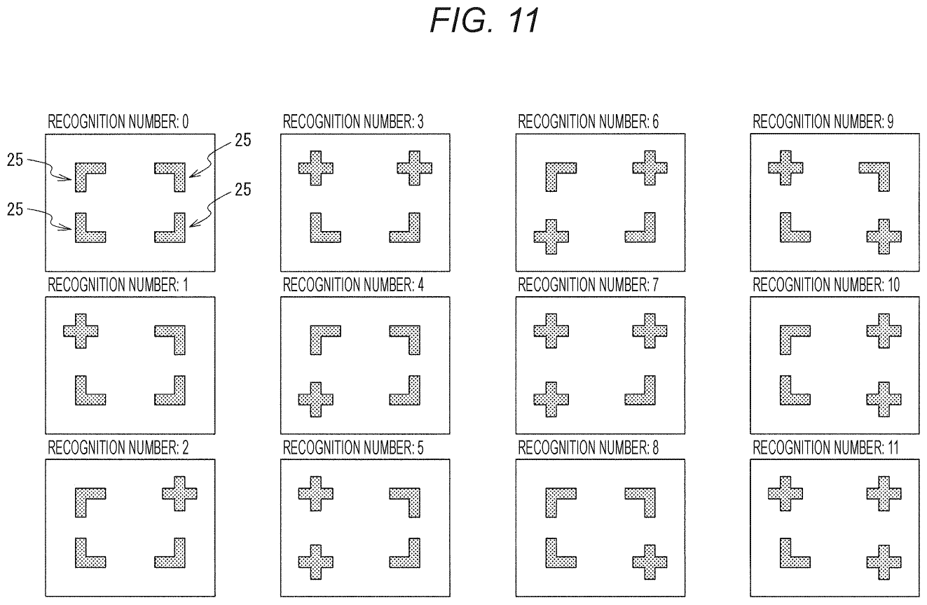

Furthermore, as illustrated in FIG. 9, positioning markers 25 are formed in a plurality of predetermined regions on the surface of the frame body 21 opposite to the rotating body 11. FIG. 9 illustrates a case where the positioning marker 25 is formed in each of the corner regions of the frame body 21. As a result, the positional deviation amount can be detected from the image data, and the floating object capturing body 20 can be positioned. As the positioning marker 25, a plurality of types of markers having different appearances is used as illustrated in FIG. 11. FIG. 11 illustrates a case where two types of markers including an L-shaped marker and a cross-shaped marker are employed as the positioning marker 25. Furthermore, as illustrated in FIG. 11, the combination pattern of the region and the type of the positioning marker 25 in the marker group including the positioning markers 25 formed in each of the plurality of regions is different for each frame body 21. As a result, the combination pattern can be associated with the identification number 24 of the frame body 21 (the floating object capturing body 20), and the identification number 24 of the floating object capturing body 20 can be determined by determining the combination pattern from the image data, and the floating object capturing body 20 can be identified. FIG. 11 is a diagram illustrating a relationship between the combination pattern of the region of the corner portion of the frame body 21 and the type of the positioning marker 25 and the identification number 24. In a case where two types of markers are employed, up to 4 bits, that is, 16 floating object capturing bodies 20 can be identified. Furthermore, FIGS. 9 and 11 illustrate a case where the frame body 21 is white and the positioning marker 25 is black.

As illustrated in FIG. 4, the imaging unit 12 is disposed in the housing 2 at a position facing an imaging region B (in a broad sense, a “second region”) on the rotation path of the floating object capturing body 20. The imaging unit 12 is located obliquely above or obliquely below the axis L in a case being viewed from an opening side of the recess 4. For example, as illustrated in FIG. 10, in a case where the floating object capturing body 20 with the identification number 24 of “0” is located in the capturing region A, a position facing the floating object capturing body 20 with the identification number 24 of “4”, “5”, “7”, or “8” can be adopted. As a result, the imaging unit 12 can be disposed at the corner portion of the housing 2, and the outer shape of the housing 2 can be minimized. In particular, since the possibility that foreign matter, rain, or the like reaches the imaging unit 12 decreases as being farther from the opening portion 5 or the drain hole 9, a position facing the floating object capturing body 20 with the identification number 24 of “4” or “5” is preferable. FIG. 4 exemplifies a case where the identification number 24 is disposed at a position facing the floating object capturing body 20 of “5” (see FIG. 10) on the upper corner side on the side surface S5 side opposite to the side surface S3. The imaging unit 12 images the floating object capturing body 20 and the frame body 21 located in the imaging region B to generate image data. The image data is output to the control unit 30. As the imaging unit 12, for example, a CMOS image sensor can be employed.

Furthermore, the optical axis of the lens of the imaging unit 12 extends in a direction orthogonal to the axis L and is positioned perpendicular to the planar floating object capturing body 20 positioned in the imaging region B. Here, in a case where the distance between the imaging unit 12 and the floating object capturing body 20 is short, the imaging unit 12 performs close-up shooting, so that the depth of field becomes narrow. Therefore, for example, in a case where the optical axis of the lens of the imaging unit 12 is inclined with respect to the planar floating object capturing body 20 located in the imaging region B, the range in focus on the floating object capturing body 20 is narrowed. On the other hand, in the present embodiment, since the optical axis of the lens of the imaging unit 12 is perpendicular to the planar floating object capturing body 20 located in the imaging region B, the range in focus on the floating object capturing body 20 can be expanded, and the entire floating object capturing body 20 can be more appropriately imaged.

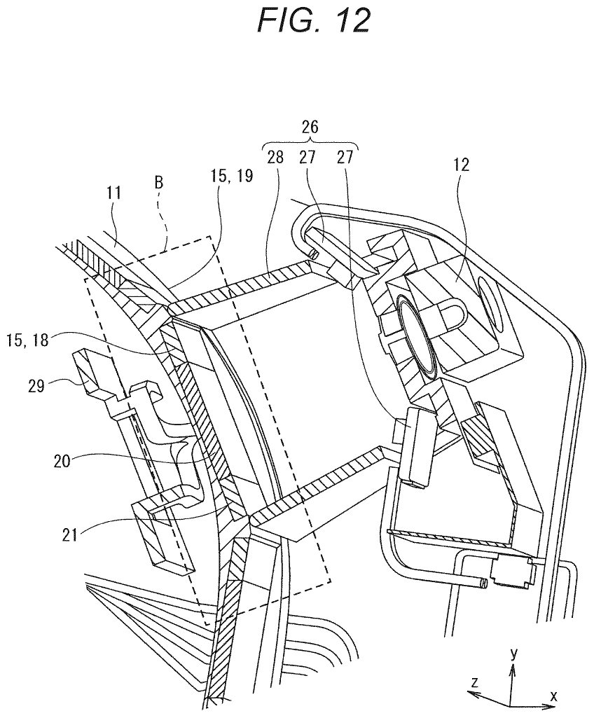

Furthermore, as illustrated in FIG. 12, an illumination mechanism 26 that illuminates the floating object capturing body 20 located in the imaging region B is disposed around the imaging unit 12. The illumination mechanism 26 includes, for example, two light emitters 27 that emit light toward the imaging region B, and an illumination box 28 that surrounds a space including the light emitter 27 and the imaging region B. The two light emitters 27 are disposed so as to sandwich the lens of the imaging unit 12, and can illuminate the floating object capturing body 20 and the frame body 21 located in the imaging region B. As the light emitter 27, for example, light emitting diode (LED) illumination can be employed. Furthermore, the illumination box 28 surrounds a space including the light emitter 27 and the imaging region B so as to block intrusion of light from the outside. As a result, it is possible to reduce the influence of the intensity and hue of external light when imaging the floating object capturing body 20. FIG. 12 illustrates a case where the illumination box 28 has a box shape having continuous openings on a side of the rotating body 11 and a side of the housing 2, the opening on the rotating body 11 side is covered with the outer peripheral surface of the rotating body 11, and the opening on the side of the housing 2 is covered with the back surface S1 of the housing 2. The width of the illumination box 28 (the length in the circumferential direction of the rotating body 11) increases from the imaging unit 12 side toward the rotating body 11 side. As a result, the light emitted from the light emitter 27 can more appropriately illuminate the floating object capturing body 20 located in the imaging region B. Furthermore, the imaging unit 12 is fixed to an end portion of the illumination box 28 on the imaging unit 12 side such that a lens of the imaging unit 12 protrudes into the illumination box 28. FIG. 12 is a diagram illustrating a cross section of the rotating body 11 broken in a plane parallel to the x-y plane in a case of being viewed from the surface S2 side.

Furthermore, the end portion of the illumination box 28 on the rotating body 11 side is in contact with a region that is the frame-shaped region 19 of the outer peripheral surface of the rotating body 11 and extends along the circumferential direction of the rotating body 11. FIG. 12 illustrates a case where the shape of the end portion of the illumination box 28 on the rotating body 11 side is an arc shape along the frame-shaped region 19 so that the rotating body 11 rotates in a state where the frame-shaped region 19 is in contact with the end portion. Furthermore, on the back surface S1 side of the housing 2, at a position overlapping the bottom portion 14 of the rotating body 11 when viewed from the back surface S1 side, a biasing portion 29 is disposed to bias the inner peripheral surface side of the rotating body 11 positioned in the imaging region B toward the outer peripheral surface side so as to press the frame-shaped region 19 against the end portion of the illumination box 28 on the rotating body 11 side. As a result, it is possible to suppress variation in the distance between the imaging unit 12 and the imaging region B due to rattling of the rotating body 11 and the shaft portion 13. Therefore, the range in focus on the floating object capturing body 20 can be expanded, and the floating object capturing body 20 can be more appropriately imaged. FIG. 12 exemplifies a case where the biasing portion 29 has an S-shaped shape in a case of being viewed from the imaging unit 12 side, and includes a spring portion in which a central portion of the S-shaped shape protrudes toward the inner peripheral surface of the rotating body 11 and is in contact with the inner peripheral surface of the rotating body 11, and a support portion fixed to the back surface S1 side of the housing 2 and indicating each of S-shaped end portions of the spring portion.

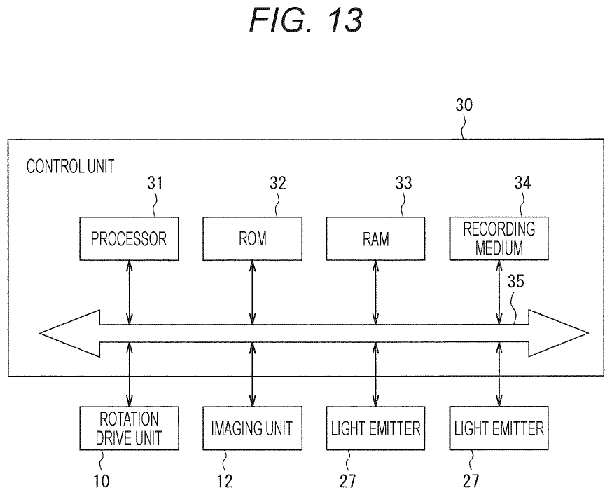

Furthermore, as illustrated in FIG. 4, the control unit 30 is disposed on the lower corner of the back surface S1 of the housing 2 on the side of the side surface S5. As illustrated in FIG. 13, the control unit 30 includes a processor 31, a ROM 32, a RAM 33, and a recording medium 34. Furthermore, in the control unit 30, each of the components 31 to 34, the imaging unit 12, and the light emitter 27 are connected to each other via a bus 35.

The processor 31 includes, for example, a processor including an arithmetic circuit such as an MPU, various processing circuits, and the like. Furthermore, the ROM 32 also stores control data such as programs and operation parameters used by the processor 31. Furthermore, the RAM 33 temporarily stores programs and the like executed by the processor 31.

Furthermore, the recording medium 34 stores data, various applications, and the like related to a floating object imaging process to be described later. As the recording medium 34, for example, a nonvolatile memory can be adopted. The floating object imaging process is a process of causing the rotation drive unit 10 to rotate the rotating body 11 and causing the imaging unit 12 to image the floating object capturing body 20 located in the imaging region B such that the floating object capturing body 20 is located in the imaging region B after being located in the capturing region A.

### 1-2 Floating Object Imaging Process

Next, a floating object imaging process executed by the processor 31 will be described.

The floating object imaging process is started when the floating object imaging device 1 is activated after the rotating body 11 is attached to the shaft portion 13 of the rotation drive unit 10.

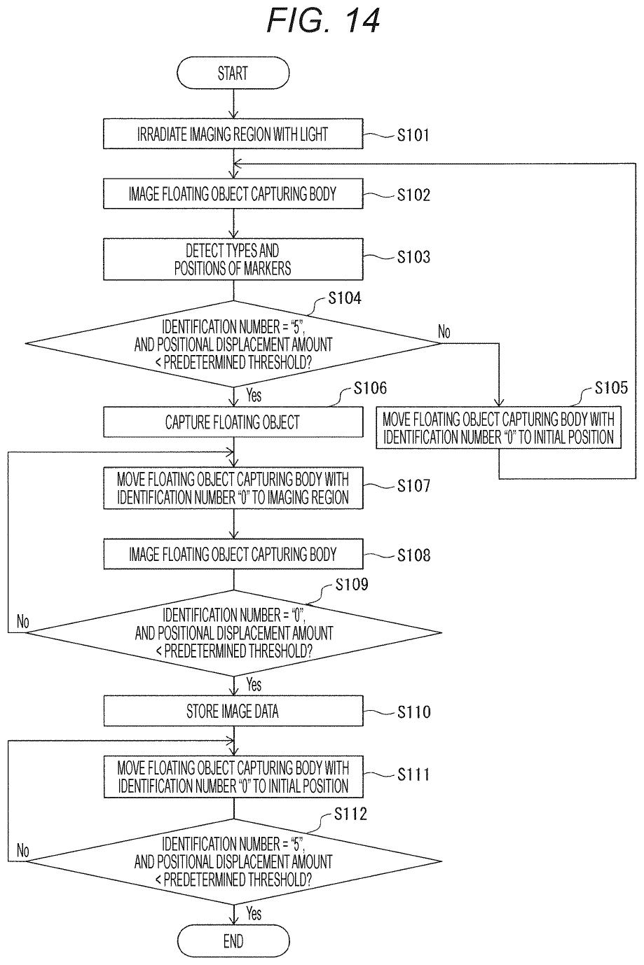

As illustrated in FIG. 14, first, in step S101, the processor 31 outputs a signal for starting light emission to the light emitter 27. As a result, the light emitter 27 starts emitting light, and the light hits the floating object capturing body 20 and the frame body 21 located in the imaging region B to illuminate the floating object capturing body 20 and the frame body 21. The light emission is continued until the floating object imaging process is ended.

Subsequently, the process proceeds to step S102, and the processor 31 outputs a signal (hereinafter, also referred to as an “imaging instruction signal”) for causing the imaging unit 12 to perform imaging to the imaging unit 12. As a result, the imaging unit 12 images the floating object capturing body 20 and the frame body 21 located in the imaging region B in the housing 2 to generate image data. The image data is output from the imaging unit 12 to the processor 31.

Subsequently, the process proceeds to step S103, and the processor 31 detects the types and positions of the four positioning markers 25 from the image data generated in step S102. As the position of the positioning marker 25, for example, coordinates in the image indicated by the image data can be adopted. For example, in a case where the positioning marker 25 has an L shape, the coordinates of the corner portion can be cited. Furthermore, for example, in a case where the positioning marker 25 has a cross shape, the coordinate of the intersection is exemplified.

Subsequently, the process proceeds to step S104, and the processor 31 detects the identification number 24 of the imaged floating object capturing body 20 on the basis of the combination pattern of the positioning markers 25 detected in step S103. Subsequently, it is determined whether the detected identification number 24 is the same as the identification number 24 selected in advance. As the identification number 24 selected in advance, for example, a numerical value obtained by adding “5” to the identification number 24 of the floating object capturing body 20 used for capturing a floating object can be adopted. Hereinafter, description will be given on the assumption that the identification number 24 of the floating object capturing body 20 used for capturing the floating object is “0”, and the identification number 24 of the floating object capturing body 20 selected in advance is “5”.

Subsequently, the processor 31 determines whether the amount of positional deviation of the positioning marker 25 from a predetermined marker target position is less than a predetermined threshold on the basis of the position of the positioning marker 25 detected in step S103. As the marker target position, for example, the position of the positioning marker 25 when the central portion of the floating object capturing body 20 coincides with the central portion of the imaging range of the imaging unit 12 can be adopted. Subsequently, in a case where the processor 31 determines that the identification number 24 is “5” and determines that the positional displacement amount of the positioning marker 25 is less than the predetermined threshold (Yes), the process proceeds to step S106. On the other hand, in other cases (No), the process proceeds to step S105.

In step S105, the processor 31 outputs a signal for rotating the rotating body 11 to the rotation drive unit 10 on the basis of the determination result of step S104 so that the central portion of the floating object capturing body 20 with the identification number 24 of “5” coincides with the central portion of the imaging range of the imaging unit 12. As a result, the rotation drive unit 10 rotates the rotating body 11 so that the floating object capturing body 20 with the identification number 24 of “0” moves to the capturing region A (initial position), and the external air that has passed through the opening portion 5 hits the floating object capturing body 20 with the identification number 24 of “0”. After the rotation of the rotating body 11 is finished, the process proceeds to step S102, and the flow of steps S102 to S104 is repeated. At this time, the rotation direction of the rotating body 11 is limited to the counterclockwise direction when viewed from the surface S2 side. As a result, the shutter 7 is moved downward and reaches the lowermost portion, thereby opening the opening 5b of the opening portion 5.

Note that the rotation direction of the rotating body 11 is based on the counterclockwise direction, but if the recessed region 18 into which foreign matter, rain, or the like has entered from the opening portion 5 does not reach the uppermost portion, there is a low possibility that the entering foreign matter, rain, or the like moves to a side of the imaging unit 12. Therefore, the rotation may be performed up to 2 positions even in the clockwise direction, that is, from the capturing region A where the floating object capturing body 20 with the identification number 24 of “0” illustrated in FIG. 10 is located to the region where the floating object capturing body 20 with the identification number 24 of “2” is located.

On the other hand, in step S106, the processor 31 waits for a predetermined time (for example, one hour) to elapse. As a result, the floating substances contained in the air that has passed through the opening portion 5 are sequentially captured by the floating object capturing body 20 with the identification number 24 of “0” for a predetermined time.

Subsequently, the process proceeds to step S107, and on the basis of the determination result of step S104 or S109, a signal for causing the rotation drive unit 10 to rotate the rotating body 11 is output to the rotation drive unit 10 so that the central portion of the floating object capturing body 20 with the identification number 24 of “0” coincides with the central portion of the imaging range of the imaging unit 12. As a result, the rotation drive unit 10 rotates the rotating body 11 so that the floating object capturing body 20 with the identification number 24 of “0” moves to the imaging region B, and the imaging unit 12 can image the floating object capturing body 20 with the identification number 24 of “0”. At this time, the rotation direction of the rotating body 11 is limited to the counterclockwise direction. That is, the processor 31 controls the rotation direction of the rotating body 11 by the rotation drive unit 10 such that the floating object capturing body 20 passes through the capturing region A, the discharge region D (“third region” in a broad sense) facing the drain hole 9, and the imaging region B in this order on the rotation path. As a result, foreign matter, rain, or the like that has entered the recessed region 18 of the rotating body 11 is discharged from the drain hole 9.

Subsequently, the process proceeds to step S108, and the processor 31 outputs an imaging instruction signal to the imaging unit 12. As a result, the imaging unit 12 images the floating object capturing body 20 (the floating object capturing body 20 having the identification number 24 of “0”) and the frame body 21 located in an imaging region B. Then, the image data (data indicating the capturing result of the floating object by the floating object capturing body 20) is output to the processor 31.

Subsequently, the process proceeds to step S109, and the processor 31 detects the combination pattern of the positioning markers 25 from the image data generated in step S108. Subsequently, on the basis of the detected combination pattern of the positioning markers 25, it is determined whether the identification number 24 of the imaged floating object capturing body 20 is “0”. Subsequently, on the basis of the detected position of the positioning marker 25, it is determined whether the amount of positional deviation of the positioning marker 25 from the marker target position is less than a predetermined threshold. Subsequently, in a case where it is determined that the identification number 24 is “0” and it is determined that the positional displacement amount of the positioning marker 25 is less than the predetermined threshold (Yes), the process proceeds to step S110. On the other hand, in other cases (No), the process proceeds to step S107.

In step S110, the processor 31 stores the image data generated in step S108 in the recording medium 34. As a result, image data of the capturing result of the floating object can be acquired.

Subsequently, the process proceeds to step S111, and the processor 31 causes the rotation drive unit 10 to rotate the rotating body 11 on the basis of the determination result of step S109 or S113 so that the central portion of the floating object capturing body 20 with the identification number 24 of “5” coincides with the central portion of the imaging range of the imaging unit 12. As a result, the rotation drive unit 10 rotates the rotating body 11 so that the floating object capturing body 20 having the identification number 24 of “0” moves to the initial position, that is, the position (capturing region A) on the side of the opening portion 5. At this time, the rotation direction of the rotating body 11 is limited to the clockwise direction as viewed from the surface S2 side. As a result, the shutter 7 is moved upward and reaches the uppermost portion, thereby closing the opening 5b of the opening portion 5.

Note that in a case where the capture and imaging of the floating object is performed using another floating object capturing body 20 subsequent to the floating object capturing body 20 with the identification number 24 of “0” described above, the rotation drive unit 10 rotates the rotating body 11 so that the another floating object capturing body 20 moves to the position (capturing region A) on the side of the opening portion 5.

Subsequently, the process proceeds to step S112, and the processor 31 outputs an imaging instruction signal to the imaging unit 12. As a result, the imaging unit 12 images the floating object capturing body 20 (the floating object capturing body 20 having the identification number 24 of “5”) located in the imaging region B and the frame body 21 to generate image data, and outputs the generated image data to the processor 31. Subsequently, the processor 31 detects a combination pattern of the positioning markers 25 from the output image data.

Subsequently, on the basis of the detected combination pattern of the positioning markers 25, it is determined whether the identification number 24 of the imaged floating object capturing body 20 is “5”. Subsequently, on the basis of the detected position of the positioning marker 25, it is determined whether the amount of positional deviation of the positioning marker 25 from the marker target position is less than a predetermined threshold. Subsequently, in a case where it is determined that the identification number 24 is “5” and it is determined that the positional displacement amount of the positioning marker 25 is less than the predetermined threshold (Yes), the floating object imaging process ends. On the other hand, in other cases (No), the process proceeds to step S111.

As described above, in the floating object imaging device 1 according to the present embodiment, the control unit 30 causes the rotation drive unit 10 to rotate the rotating body 11 and causes the imaging unit 12 to image the floating object capturing body 20 located in the imaging region B such that the floating object capturing body 20 is located in the imaging region B after being located in the capturing region A. As a result, after the floating object capturing body 20 captures the floating object floating in the air in the capturing region A, the floating object capturing body 20 is moved to the imaging region B, and in the imaging region B, the floating object capturing body 20 capturing the floating object can be imaged by the imaging unit 12. Therefore, it is possible to provide the floating object imaging device 1 capable of capturing and imaging a floating object by one device.

Here, the opening portion 5 requires air to be vertically applied to the floating object capturing body 20. Furthermore, in a case of considering capturing and imaging of a floating object by one device, the distance between the imaging unit 12 and the floating object capturing body 20 is close, and it is essential for the imaging unit 12 to perform close-up shooting. Therefore, the depth of field in the imaging unit 12 becomes narrow. Therefore, the imaging unit 12 is required to image the floating object capturing body 20 from the vertical direction. Therefore, it is necessary to achieve both vertical application of air to the floating object capturing body 20 and vertical imaging of the floating object capturing body 20.

On the other hand, in the floating object imaging device 1 according to the present embodiment, the floating object capturing body 20 is disposed on the outer peripheral surface of the rotating body 11, the opening portion 5 is formed at a position facing the capturing region A on the rotation path, and the imaging unit 12 is disposed at a position facing the imaging region B. As a result, the capturing place of the floating object and the capturing place of the captured floating object can be separated. Therefore, it is possible to achieve both vertical application of air to the floating object capturing body 20 and vertical imaging of the floating object capturing body 20.

### 1-3 Modifications

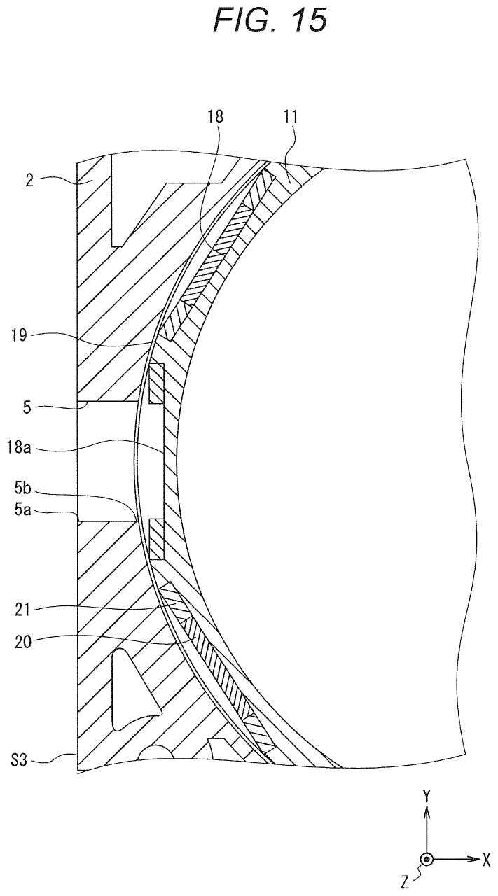

(1) Note that, in the present embodiment, an example in which the shutter mechanism 6 is used to prevent entry of foreign matter, rain, or the like into the recessed region 18 of the rotating body 11 has been described, but other configurations may be adopted. For example, the shutter mechanism 6 may be omitted, and a specific recessed region 18a of the plurality of recessed regions 18 of the rotating body 11 may function as a portion that receives foreign matter, rain, or the like when neither capturing nor capturing an image of a floating object is performed. As an example, when the control unit 30 does not capture or image a floating object, as illustrated in FIG. 15, the rotation drive unit 10 drives the rotating body 11 so that the predetermined specific recessed region 18a is located in the capturing region A. As a result, it is possible to prevent the floating object capturing body 20 in the other recessed regions 18 from coming into contact with foreign matter, rain, or the like, to prevent deterioration in quality of the floating object capturing body 20, and to simplify the configuration of the floating object imaging device 1. In the predetermined recessed region 18a, since the floating object is not captured, the floating object capturing body 20 is omitted.

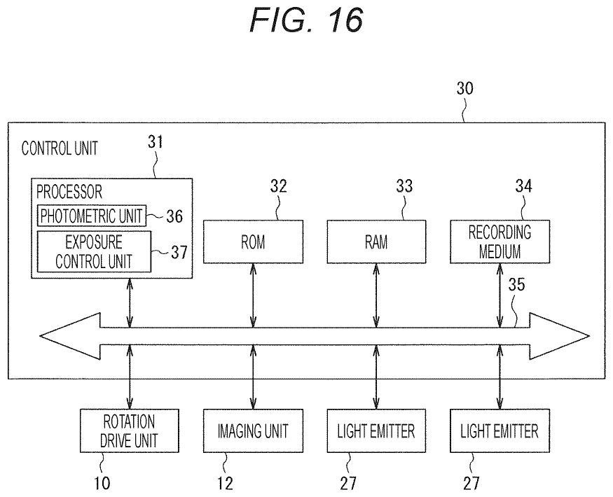

(2) Furthermore, in the present embodiment, an example in which the identification number 24 is detected and positioned using the positioning marker 25 has been described, but other configurations can also be adopted. For example, the exposure condition may be controlled using the positioning marker 25. As an example, as illustrated in FIG. 16, a photometric unit 36 that acquires luminance information of a region including the frame body 21 and the positioning marker 25, and an exposure control unit 37 that controls an exposure condition at the time of performing imaging with respect to the imaging unit 12 on the basis of the luminance information acquired by the photometric unit 36 are included. As a result, the imaging unit 12 images the floating object capturing body 20 to generate image data, the photometric unit 36 acquires luminance information from the generated image data, the exposure control unit 37 controls the exposure condition, and then the imaging unit 12 again images the floating object capturing body 20. FIG. 16 illustrates a case where the photometric unit 36 and the exposure control unit 37 are implemented by the processor 31. Furthermore, as illustrated in FIG. 9, one of the frame body 21 and the positioning marker 25 is white, and the other is black. FIG. 9 illustrates a case where the frame body 21 is white and the positioning marker 25 is black. Thus, for example, the exposure condition is controlled based on the luminance information of the region including white and black, so that the exposure condition can be more appropriately controlled. As the exposure condition, for example, a shutter speed and an ISO value can be adopted.

(3) Furthermore, in the present embodiment, the example in which the axis L serving as the rotation center of the rotating body 11 extends in the horizontal direction has been described, but other configurations can also be adopted. For example, the axis L may extend in a direction inclined from the horizontal direction, such as a vertical direction (a direction in which gravity acts).

Note that, the present technology can also have the following configurations.

(1)

A floating object imaging device including:
a housing including a recess;
a rotating body that is disposed within the recess and rotates about an axis extending in a depth direction of the recess;
a plurality of floating object capturing bodies including a material capable of capturing a floating object floating in air and disposed on an outer peripheral surface of the rotating body at intervals in a circumferential direction;
a rotation drive unit that is disposed in the housing and rotates the rotating body about the axis;
an opening portion formed in the housing at a position facing a first region on a rotation path of the floating object capturing bodies so that the floating object capturing bodies located in the first region are exposed to an outside;
an imaging unit disposed in the housing at a position facing a second region on the rotation path of the floating object capturing bodies so that the floating object capturing bodies located in the second region are imaged; and
a control unit that causes the rotation drive unit to rotate the rotating body so that the floating object capturing bodies are located in the second region after being located in the first region, and causes the imaging unit to image the floating object capturing bodies located in the second region.

(2)

The floating object imaging device according to (1) described above, in which
the recess includes a circular recess,
the rotating body includes a cylindrical outer peripheral portion, and
an outer peripheral surface of the outer peripheral portion includes a plurality of recessed regions formed at intervals in the circumferential direction and a frame-shaped region surrounding the recessed regions, and the floating object capturing bodies are disposed in the recessed regions.

(3)

The floating object imaging device according to (2) described above, in which each of the floating object capturing bodies has a flat plate shape having a normal line extending in a direction orthogonal to the axis, and
an opening direction of the opening portion and an optical axis of a lens of the imaging unit extend in a direction orthogonal to the axis.

(4)

The floating object imaging device according to (3) described above, in which
the axis extends in one horizontal direction,
the opening direction of the opening portion extends in another horizontal direction orthogonal to the one horizontal direction,
the floating object imaging device includes a through hole penetrating from a bottom side of an inner peripheral surface of the recess to an outside of the housing, and
the control unit controls a rotation direction of the rotating body by the rotation drive unit such that the floating object capturing bodies pass through the rotation path in order of the first region, a third region facing the through hole, and the second region.

(5)

The floating object imaging device according to (4) described above, in which
the imaging unit is located obliquely upward or obliquely downward with respect to the axis in a case of being viewed from an opening side of the recess.

(6)

The floating object imaging device according to any one of (1) to (5) described above, in which
the rotating body is configured to be attachable to and detachable from a shaft portion of the rotation drive unit.

(7)

The floating object imaging device according to any one of (1) to (6) described above, in which
a plurality of the floating object capturing bodies includes a plurality of types of floating object capturing bodies having different materials from each other.

(8)

The floating object imaging device according to any one of (1) to (7) described above, further including
an illumination mechanism that illuminates the floating object capturing bodies located in the second region.

(9)

The floating object imaging device according to (8) described above, in which
the illumination mechanism includes a light emitter that emits light toward the second region, and an illumination box that surrounds a space including the light emitter and the second region.

(10)

The floating object imaging device according to (9) described above, in which
the rotating body includes a cylindrical outer peripheral portion,
an outer peripheral surface of the outer peripheral portion includes a plurality of recessed regions formed at intervals in the circumferential direction and a frame-shaped region surrounding the recessed regions, and the floating object capturing bodies are disposed in the recessed regions, and
the floating object imaging device further includes a biasing portion that biases an inner peripheral surface side of the rotating body located in the second region toward an outer peripheral surface side so as to press the frame-shaped region against an end portion of the illumination box on a side of the rotating body.

(11)

The floating object imaging device according to any one of (1) to (10) described above, further including
a shutter mechanism that opens and closes the opening portion in conjunction with rotation of the rotating body.

(12)

The floating object imaging device according to (11) described above, in which
the rotating body includes a cylindrical outer peripheral portion,
an outer peripheral surface of the outer peripheral portion includes a plurality of recessed regions formed at intervals in the circumferential direction and a frame-shaped region surrounding the recessed regions, and the floating object capturing bodies are disposed in the recessed regions, and
the shutter mechanism includes a shutter that slides and moves in the circumferential direction on an inner peripheral surface of the recess, and a friction component that is attached to a surface of the shutter on a side of the rotating body and comes into contact with the frame-shaped region of the rotating body.

(13)

The floating object imaging device according to (12) described above, in which
a radius of an inner peripheral surface of a region where the shutter slides and moves in the inner peripheral surface of the recess is larger than a radius of the inner peripheral surface of other regions.

(14)

The floating object imaging device according to any one of (1) to (10) described above, in which
the rotating body includes a cylindrical outer peripheral portion,
an outer peripheral surface of the outer peripheral portion includes a plurality of recessed regions formed at intervals in the circumferential direction, and
when neither capturing nor imaging the floating object, the control unit causes the rotation drive unit to rotate the rotating body such that a predetermined specific recessed region is located in the first region.

(15)

The floating object imaging device according to any one of (1) to (14) described above, further including
a frame body that surrounds a periphery of each of the floating object capturing bodies,
in which a positioning marker is formed in each of a plurality of predetermined regions on a surface of the frame body on a side opposite to the rotating body.

(16)

The floating object imaging device according to (15) described above, in which
a plurality of types of markers having different appearances is used as the positioning marker, and
in a marker group including the positioning marker formed in each of a plurality of the regions, a combination pattern of the region and a type of the positioning marker is different for each of the frame bodies.

(17)

The floating object imaging device according to (15) or (16) described above, further including:
a photometric unit that acquires luminance information of a region including the frame body and the positioning marker, one of the frame body and the positioning marker being white and another of the frame body and the positioning marker being black; and
an exposure control unit that controls an exposure condition when imaging is performed with respect to the imaging unit on the basis of the luminance information acquired by the photometric unit.

### REFERENCE SIGNS LIST

1 Floating object imaging device
2 Housing
3 Back lid
4 Recess
5 Opening portion
5a, 5b Opening
6 Shutter mechanism
7 Shutter
7a Opening portion
8 Friction component
9 Drain hole
10 Rotation drive unit
11 Rotating body
12 Imaging unit
13 Shaft portion
14 Bottom portion
15 Outer peripheral portion
16 Through hole
17 Lid
18, 18a Recessed region
19 Frame-shaped region
20 Floating object capturing body
21 Frame body
22 End portion
23 Positioning hole
24 Identification number
25 Positioning marker
26 Illumination mechanism
27 Light emitter
28 Illumination box
29 Biasing portion
30 Control unit
31 Processor
32 ROM
33 RAM
34 Recording medium
35 Bus
36 Photometric unit
37 Exposure control unit

What is claimed is:

1. A floating object imaging device, comprising:
a housing including a recess;
a rotating body that is disposed within the recess and rotates about an axis extending in a depth direction of the recess;
a plurality of floating object capturing bodies including a material capable of capturing a floating object floating in air and disposed on an outer peripheral surface of the rotating body at intervals in a circumferential direction;
a rotation drive unit that is disposed in the housing and rotates the rotating body about the axis;
an opening portion formed in the housing at a position facing a first region on a rotation path of the floating object capturing bodies so that the floating object capturing bodies located in the first region are exposed to an outside;
an imaging unit disposed in the housing at a position facing a second region on the rotation path of the floating object capturing bodies so that the floating object capturing bodies located in the second region are imaged; and
a control unit that causes the rotation drive unit to rotate the rotating body so that the floating object capturing bodies are located in the second region after being located in the first region, and causes the imaging unit to image the floating object capturing bodies located in the second region.

2. The floating object imaging device according to claim 1, wherein
the recess includes a circular recess,
the rotating body includes a cylindrical outer peripheral portion, and
an outer peripheral surface of the outer peripheral portion includes a plurality of recessed regions formed at intervals in the circumferential direction and a frame-shaped region surrounding the recessed regions, and the floating object capturing bodies are disposed in the recessed regions.

3. The floating object imaging device according to claim 2, wherein
each of the floating object capturing bodies has a flat plate shape having a normal line extending in a direction orthogonal to the axis, and
an opening direction of the opening portion and an optical axis of a lens of the imaging unit extend in a direction orthogonal to the axis.

4. The floating object imaging device according to claim 3, wherein
the axis extends in one horizontal direction,
the opening direction of the opening portion extends in another horizontal direction orthogonal to the one horizontal direction,
the floating object imaging device includes a through hole penetrating from a bottom side of an inner peripheral surface of the recess to an outside of the housing, and
the control unit controls a rotation direction of the rotating body by the rotation drive unit such that the floating object capturing bodies pass through the rotation path in order of the first region, a third region facing the through hole, and the second region.

5. The floating object imaging device according to claim 4, wherein
the imaging unit is located obliquely upward or obliquely downward with respect to the axis in a case of being viewed from an opening side of the recess.

6. The floating object imaging device according to claim 1, wherein
the rotating body is configured to be attachable to and detachable from a shaft portion of the rotation drive unit.

7. The floating object imaging device according to claim 1, wherein
a plurality of the floating object capturing bodies includes a plurality of types of floating object capturing bodies having different materials from each other.

8. The floating object imaging device according to claim 1, further comprising an illumination mechanism that illuminates the floating object capturing bodies located in the second region.

9. The floating object imaging device according to claim 8, wherein
the illumination mechanism includes a light emitter that emits light toward the second region, and an illumination box that surrounds a space including the light emitter and the second region.

10. The floating object imaging device according to claim 9, wherein
the rotating body includes a cylindrical outer peripheral portion,
an outer peripheral surface of the outer peripheral portion includes a plurality of recessed regions formed at intervals in the circumferential direction and a frame-shaped region surrounding the recessed regions, and the floating object capturing bodies are disposed in the recessed regions, and
the floating object imaging device further includes a biasing portion that biases an inner peripheral surface side of the rotating body located in the second region toward an outer peripheral surface side so as to press the frame-shaped region against an end portion of the illumination box on a side of the rotating body.

11. The floating object imaging device according to claim 1, further comprising
a shutter mechanism that opens and closes the opening portion in conjunction with rotation of the rotating body.

12. The floating object imaging device according to claim 11, wherein
the rotating body includes a cylindrical outer peripheral portion,
an outer peripheral surface of the outer peripheral portion includes a plurality of recessed regions formed at intervals in the circumferential direction and a frame-shaped region surrounding the recessed regions, and the floating object capturing bodies are disposed in the recessed regions, and
the shutter mechanism includes a shutter that slides and moves in the circumferential direction on an inner peripheral surface of the recess, and a friction component that is attached to a surface of the shutter on a side of the rotating body and comes into contact with the frame-shaped region of the rotating body.

13. The floating object imaging device according to claim 12, wherein
a radius of an inner peripheral surface of a region where the shutter slides and moves in the inner peripheral surface of the recess is larger than a radius of the inner peripheral surface of other regions.

14. The floating object imaging device according to claim 1, wherein
the rotating body includes a cylindrical outer peripheral portion,
an outer peripheral surface of the outer peripheral portion includes a plurality of recessed regions formed at intervals in the circumferential direction, and
when neither capturing nor imaging the floating object, the control unit causes the rotation drive unit to rotate the rotating body such that a predetermined specific recessed region is located in the first region.

15. The floating object imaging device according to claim 1, further comprising
a frame body that surrounds a periphery of each of the floating object capturing bodies,
wherein a positioning marker is formed in each of a plurality of predetermined regions on a surface of the frame body on a side opposite to the rotating body.

16. The floating object imaging device according to claim 15, wherein
a plurality of types of markers having different appearances is used as the positioning marker, and
in a marker group including the positioning marker formed in each of a plurality of the regions, a combination pattern of the region and a type of the positioning marker is different for each of the frame bodies.

17. The floating object imaging device according to claim 15, further comprising:
a photometric unit that acquires luminance information of a region including the frame body and the positioning marker, one of the frame body and the positioning marker being white and another of the frame body and the positioning marker being black; and
an exposure control unit that controls an exposure condition when imaging is performed with respect to the imaging unit on a basis of the luminance information acquired by the photometric unit.
